(19) United States
(12) Patent Application Publication
(10) Pub. No.: US 2011/0268748 A1
(43) Pub. Date: Nov. 3, 2011

(54) NURR-1 Interacting Protein (NuIP)

(75) Inventor: Howard J. Federoff, Bethesda, MD (US)

(73) Assignee: The University of Rochester, Rochester, NY (US)

(21) Appl. No.: 13/063,143

(22) PCT Filed: Sep. 9, 2009
(86) PCT No.: PCT/US09/56346

§ 371 Date: Jul. 1, 2011

Related U.S. Application Data
(62) Division of application No. 61/095,540, filed on Sep. 9, 2008.

(51) Int. Cl. A61K 39/395; C12N 5/0793; G01N 33/566; C12Q 1/02; C12Q 1/68; A61K 38/10; C07K 7/08; C07K 16/18; C07H 21/04; A61P 25/00; A61P 25/16; C12N 5/071; A61K 31/713

(52) U.S. Cl. 424/172.1; 435/375; 436/501; 435/29; 435/6.13; 514/44 A; 530/326; 530/387.9; 536/23.5; 514/17.7; 514/21.4

(57) ABSTRACT

Provided herein are methods of promoting the activity of Nurr1 in a cell comprising contacting the cell with NuIP or an analog or fragment thereof. Also provided are methods of treating or preventing a condition associated with reduced dopaminergic function in a subject, comprising administering to the subject NuIP or an analog or fragment thereof. Methods of inhibiting the activity of Nurr1 in a cell comprising contacting the cell with a NuIP inhibitor are provided. Methods of screening for agents that modulate the interaction of Nurr1 and NuIP are also provided.

### CROSS-REFERENCE TO RELATED APPLICATION

The present application claims priority to U.S. Provisional Application No. 61/095,540, filed Sep. 9, 2008, which is incorporated by reference herein in its entirety.

### STATEMENT REGARDING FEDERALLY FUNDED RESEARCH

This invention was made with government support under Grant No. DAMD17-02-1-0695 awarded by the Department of Defense. The government has certain rights in the invention.

### BACKGROUND

Development of mesencephalic dopaminergic neurons in mice requires the expression of the transcription factor Nurr1 (also known as NR4A2). Loss of Nurr1 function through gene targeting results in the failure of midbrain progenitors to complete specification of the dopaminergic lineage (Zetterstrom et al., Science 276:248-50 (1997)). Nurr1, an orphan member of the nerve growth factor inducible-B subfamily of nuclear receptors (NRs), has no known activating ligand. Recent crystallographic analysis of the Nurr1 ligand-binding domain (NLBD) demonstrates a ligand pocket that is apparently too confining to accommodate lipophilic ligands, such as steroids (Wang et al., Nature 423:555-60 (2003)). This does not address how the transcriptional activity of Nurr1 is regulated. Among the mechanisms that may contribute to modulating its transcriptional activity include posttranslational modification and/or interaction with other proteins that induce the adoption of an “activated” NLBD structure. Indeed, studies on the assembly of an active NLBD in HEK293 cells has implicated c-ret signaling as a negative regulator (Wang et al., 2003). With the exception of formation of transactivating heterodimeric complexes with other NRs, such as retinoid-X receptor α (RXRα) (Perlmann and Jansson, Genes Dev. 9:769-82 (1995)), all other interactors identified thus far, such as p57kip2 and PIASγ (Joseph et al., Proc. Natl. Acad. Sci. USA 100:15619-24 (2003); Galleguillos et al., J. Biol. Chem. 279:2005-11 (2004)), are negative regulators of Nurr1 transcriptional activity.

### SUMMARY

Provided herein are methods of promoting the activity of Nurr1 in a cell. The methods comprise contacting the cell with Nurr1-Interacting Protein (NuIP) or an analog or fragment thereof.

Also provided are methods of treating or preventing a condition associated with reduced dopaminergic function in a subject. The methods comprise administering to the subject NuIP or an analog or fragment thereof.

Methods of inhibiting the activity of Nurr1 in a cell comprising contacting the cell with a NuIP inhibitor are provided.

Polypeptides are provided comprising less than 1093 amino acids and comprising the amino acid sequence CVMDGWPGEADKPSRA (SEQ ID NO:3). Also provided are antibodies that bind the polypeptide, nucleic acids encoding the polypeptide and compositions comprising the polypeptide.

Further provided are methods of screening for agents that modulate the interaction of Nurr1 and NuIP. For example, the method includes the steps of providing a composition comprising Nurr1 and NuIP, contacting the composition with an agent to be tested, and determining whether the agent to be tested modulates the interaction of Nurr1 and NuIP. By way of another example, the method includes the steps of providing a population of cells, wherein the cells express Nurr1 and NuIP, contacting the cells with an agent to be tested, and determining whether the agent to be tested modulates the interaction of Nurr1 and NuIP.

### DESCRIPTION OF DRAWINGS

FIG. 1 shows a schematic of Nurr1 bait constructs used in the yeast two-hybrid assay. Different forms of Nurr1 cDNA were cloned in-frame with the Gal4 DNA-binding domain and were subsequently tested in a yeast two-hybrid assay. Gal4Nurr1 contained the full length Nurr1 (SEQ ID NO:31); Gal4NLBD contained the Nurr1 ligand binding domain (SEQ ID NO:32); Gal4NLDBA583 contained the Nurr1 ligand binding domain with the AF2 domain deleted (SEQ ID NO:33); Gal4Nurr1 589A contained the full length Nurr1 protein with a substitution of an alanine for an aspartic acid at amino acid number 589 (SEQ ID NO:34); and Gal4NLBD 589A contained the Nurr1 ligand binding domain with a substitution of an alanine for an aspartic acid at amino acid number 589 (SEQ ID NO:35).

FIGS. 2A and 2B show alternative splicing of the NuIP gene and the tissue distribution of the different transcript isoforms. FIG. 2A shows a schematic representation of the putative alternatively spliced isoforms of the NuIP gene. FIG. 2B shows representative gels of RT-PCR assays using primers specific for the alternatively spliced NuIP transcripts, Nurr1, and GAPDH. The RT-PCR assays were performed on mRNAs extracted from different mouse tissues. Nurr1 and full-length NuIP transcripts were consistently co-expressed in the various tissues examined. Lane 1: Midbrain; Lane 2: Cortex; Lane 3: Spleen; Lane 4: Kidney; Lane 5: Heart; Lane 6: Striatum; Lane 7: Cerebellum; Lane 8: Pons/Medulla; Lane 9: Eye; and Lane 10: VM (E13.5).

FIG. 3 shows the NuIP ORF. The conceptual amino acid sequence (SEQ ID NO:1) of the longest ORF encoded by a full-length NuIP transcript (SEQ ID NO:2).

FIG. 4 shows a graph demonstrating that NuIP interacts with full-length NLBD and NLBD583. The interaction between NuIP and Nurr1 was confirmed by a mammalian two-hybrid assay. MN9D cells were transfected with a luciferase reporter, which was driven by five UAS Gal4-binding sites, and bait constructs (Gal4DB, GAL4NLBD, or GAL4NLBD583). Putative interacting constructs (VP16, control; VP16-NuIP) were also cotransfected to determine whether they would stimulate transcription by bringing together the TAD of VP16 with the GAL4 DNA binding domain of the bait constructs. Data are expressed as average relative units±SD. Significant differences (*p<0.01) were observed in the presence of VP16-NuIP using Student's t test. RLU: Relative light units.

FIGS. 5A-5C show coimmunoprecipitation of NuIP and Nurr1. MN9D cells were cotransfected with a NuIP-V5 expression vector and a Flag-Nurr1 expression vector. Twenty four hours after transfection, nuclear extracts were prepared by NE-PER nuclear extraction reagent (Pierce Chemical; Rockford, Ill.) and subjected to coimmunoprecipitation. FIGS. 5A and 5B show images of Western blots demonstrating that NuIP-V5 was immunoprecipitated and detected with a monoclonal V5 antibody (FIG. 5A). The Flag-Nurr1 protein coimmunoprecipitated with the NuIP-V5 protein and was detected with a monoclonal anti-Flag antibody (FIG. 5B). Arrows indicate the migration positions of NuIP-V5 and Flag-Nurr1 proteins. To test the interaction of endogenous Nurr1 and NuIP protein, mouse SN tissue lysates were incubated with rabbit anti-Nurr1 antibody or no antibody control and pulled down by Protein A/G beads. FIG. 5C shows an image of a Western blot demonstrating that the endogenous Nurr1 and NuIP protein interact. The immunoprecipitated proteins were separated by SDS-PAGE gel and blotted by a rabbit anti-NuIP protein. Arrows indicate the migration position of NuIP protein.

FIG. 6 shows a graph demonstrating NuIP potentiates the activity of Nurr1 on NBRE. MN9D cells were cotransfected with a NBRE-containing luciferase reporter construct and Nurr1 or a NBRE-containing luciferase reporter construct, Nurr1, and NuIP. Cell extracts were subsequently assayed for luciferase activity in triplicate samples. The data are presented as mean values±SD, and the experiments were repeated three times with similar results. *p<0.05 compared with Nurr1 alone using Student's t test. RLU: Relative light units.

FIG. 7 shows a graph demonstrating NuIP augments Nurr1 transcriptional activity on the TH promoter in MN9D cells. MN9D cells were transfected with different length TH promoter constructs (TH3, TH6, and TH9 kb promoters driving β-galactosidase) together with either expression vectors encoding Nurr1, NuIP, or Nurr1 and NuIP to determine which proteins would stimulate TH promoter-reporter gene expression. Nurr1 stimulated transcription of the 3 kb, 6 kb, and 9 kb promoter-reporter constructs. Addition of NuIP augments Nurr1-dependent transcription. NuIP transfection alone produced no stimulation. The data are presented as mean values±SD for triplicate samples, and the experiments were repeated three times with similar results. *p<0.05 compared with Nurr1 alone using Student's t test. RLU: Relative light units.

FIGS. 8A and 8B show NuIP potentiates the assembly of H1 and H3-12 domains of NLBD. The ability of NuIP to augment the assembly of H1 and H3-12 of the NLBD was examined using an assembly assay in HEK293 cells. Cells were transfected with Gal4H1 and either VP16 alone or VP16H3-12 in the presence or absence of NuIP. Addition of NuIP construct further augments the assembly of the NLBD (*p<0.001, Student's t test; mean±SD). Each condition consisted of triplicate samples, and the experiments were repeated three times with similar results. RLU: Relative light units.

FIGS. 9A-9C show coimmunolocalization of NuIP with TH in adult substantia nigra. A NuIP specific antibody was developed, tested, and used to detect NuIP protein expression. FIG. 9A shows an image of a Western blot of cell lysates from a clonal MN9D cell line that were transfected with either HSVlacZ (lane 1) or HSVNuIP (lane 2). Lanes 3 and 4 were incubated with a NuIP antibody solution that has been preincubated with a NuIP peptide. FIG. 9B shows an image of a Western blot for β-actin of the same cell lysates described above demonstrating equivalent protein loading. FIG. 9C shows immunofluorescent images of adult mice (3 months old; n=3) midbrains sectioned and immunostained with anti-NuIP (FIG. 9C, sections a and d) and anti-TH (FIG. 9C, sections b and e). Each primary antibody was developed with a different fluorophore coupled secondary antibody. Sections were visualized by confocal microscopy at the level of the substantia nigra. Colocalization of expression in dopaminergic neurons is shown in the overlay channel (FIG. 9C, sections c and fat higher magnification).

FIGS. 10A-10E show the effects of NuIP knockdown in an engineered MN9D cell line. FIG. 10A shows the domain structure of NuIP and sites targeted in the corresponding mRNA by inducibly expressed siRNA. RUN domain: amino acids (aa) 44-189 (SEQ ID NO:29); TBC domain: aa 881-1053 (SEQ ID NO:30). siRNA generated from RNAi#1 vector targets the junction of exons 12 and 13, and siRNA from RNAi#2 targets exon 8. FIG. 10B shows images of Western blots demonstrating inducible knockdown of NuIP protein. Stable MN9D cells expressing the tetracycline repressor were transfected with pSUPERIOR constructs with or without siDNA inserts. Twenty-four hours after transfection, 2 μg/ml of DOX was added to induce siRNA expression. The cells were harvested 72 hours after induction. Cell lysates were analyzed for NuIP, DAT, TH, and β-tubulin. FIG. 10C shows a graph demonstrating inducible knockdown of NuIP mRNA. NuIP transcript levels were quantified with qRT-PCR and normalized to 18S ribosomal RNA; averages of triplicate quantification are shown. Error bars indicate SD. FIG. 10D shows a graph demonstrating MN9D cell numbers after inducible RNAi knockdown of NuIP mRNA. The results represent the mean of seven independent experiments; error bars indicate SD. FIG. 10E shows a graph demonstrating the downregulation of DAT in stably transfected MN9Dcells in which NuIP was knocked-down. Cells grown as above were harvested 72 hours after RNAi induction, protein lysates were prepared, samples were analyzed via Western blots, lanes were scanned, and the intensity of DAT and TH expression was determined. A significant difference was observed between mock and RNAi for DAT (p=0.006). The results shown represent the mean of 2-4 independent experiments. Error bars indicate SD.

### DETAILED DESCRIPTION

As described herein, a protein that interacts and modulates the action of Nurr1 in the developing midbrain was identified. Screening a yeast two-hybrid library prepared from developing mouse embryonic mesencephalon for Nurr1 ligand-binding domain (NLBD) interactors resulted in the identification of a new family of gene products that interact with and regulate the activity of Nurr1. This family of gene products arises from alternative splicing from a single gene, among which the longest product was termed the Nurr1-interacting protein (NuIP). Using a mammalian two-hybrid assay in the MN9D dopaminergic cell line, NuIP was shown to interact with both the full-length NLBD and the AF2-deleted NLBD, showing it interacts with Nurr1 by a mechanism dissimilar from other known modulators such as RXRα. Interaction of Nurr1 and NuIP was further demonstrated by coimmunoprecipitation in MN9D cells and endogenous substantia nigra (SN) lysates. When coexpressed with Nurr1, NuIP protein potentiates the transcriptional activity of Nurr1 on both a nerve growth factor inducible-B response element (NBRE)-containing reporter construct and an endogenous tyrosine hydroxylase (TH) promoter reporter construct. To test the mechanism by which NuIP regulates Nurr1 activity, an NLBD assembly assay was performed and demonstrated that NuIP can promote the assembly of NLBD. Using a polyclonal antibody generated against an NuIP peptide, it was shown that NuIP was extensively colocalized with Nurr1 in adult midbrain dopaminergic neurons. Finally, to evaluate the endogenous function of NuIP in dopaminergic cells, the expression of NuIP gene was suppressed in MN9D cells by small interfering RNA (siRNA). It was observed that loss of NuIP function led to decreased cell number in culture and decreased expression of a Nurr1 target gene, the dopamine transporter (DAT). Together, these results show NuIP interacts and positively regulates the activity of Nurr1 protein possibly by promoting the assembly of the NLBD.

Provided herein is a method of promoting the activity of Nurr1 in a cell comprising contacting the cell with NuIP or an analog or fragment thereof. Optionally, the promoted activity is expression of a Nurr1 target gene. The gene is, for example, tyrosine hydroxylase or a nerve growth factor inducible gene. Optionally, the cell is a dopaminergic neuron. The promoted activity is, for example, an increase in cell proliferation.

Also provided is a method of treating or preventing a condition associated with reduced dopaminergic function in a subject, comprising administering to the subject NuIP or an analog or fragment thereof. Optionally, the condition associated with reduced dopaminergic function is Parkinson's Disease, dementia with Lewy body, diffuse Lewy body with Parkinsons's Disease or attention deficit disorder. Optionally, the method includes the step of selecting a subject with or at risk of developing the condition, such as Parkinson's Disease. Optionally, the subject at risk of developing the condition has a history of head trauma, a family history of the condition (e.g., Parkinson's Disease), or early signs and symptoms (e.g., resting tremor) associated with the condition.

Also provided is a method of inhibiting the activity of Nurr1 in a cell comprising contacting the cell with a NuIP inhibitor. Optionally, the NuIP inhibitor is a NuIP siRNA molecule. Optionally, the NuIP siRNA molecule targets SEQ ID NO:27. Optionally, the NuIP siRNA molecule targets SEQ ID NO:28.

A 21-25 nucleotide NuIP siRNA sequence can, for example, be produced from an expression vector by transcription of a short-hairpin RNA (shRNA) sequence, a 60-80 nucleotide precursor sequence, which is subsequently processed by the cellular RNAi machinery to produce a siRNA sequence. Alternatively, a 21-25 nucleotide siRNA sequence can, for example, be synthesized chemically. Chemical synthesis of siRNA sequences is commercially available from such corporations as Dharmacon, Inc. (Lafayette, Colo.), Qiagen (Valencia, Calif.), and Ambion (Austin, Tex.). A siRNA sequence preferably binds a unique sequence within the NuIP mRNA with exact complementarity and results in the degradation of the NuIP mRNA molecule. A siRNA sequence can bind anywhere within the NuIP mRNA molecule. Optionally, the NuIP siRNA can target the sequence 5′-GUACCAGAUCCUCUCCAGA-3′ (SEQ ID NO:27) corresponding to nucleotides 1464-1482 of the mouse NuIP mRNA nucleotide sequence, wherein position 1 begins with the first nucleotide of the coding sequence of the NuIP mRNA molecule at Accession Number NM_{—}172718 at www.pubmed.gov. Optionally, the NuIP siRNA can target the sequence 5′-CCCGGGACCUCGUGCAUAA-3′ (SEQ ID NO:28) corresponding to nucleotides 3236-3254 of the mouse NuIP mRNA nucleotide sequence. Methods of delivering siRNA molecules are known in the art, e.g., see Oh and Park, Adv. Drug Deliv. Rev. 61(10):850-62 (2009); Gondi and Rao, J. Cell. Physiol. 220(2):285-91 (2009); and Whitehead et al., Nat. Rev. Drug Discov. 8(2):129-38 (2009).

Provided for use in the methods and compositions herein are a NuIP, a NuIP analog, a NuIP fragment, a NuIP analog fragment, and variants or isoforms of a NuIP or a NuIP analog. Optionally, the NuIP analog is, for example, an agonistic antibody to Nurr1 or a small molecule.

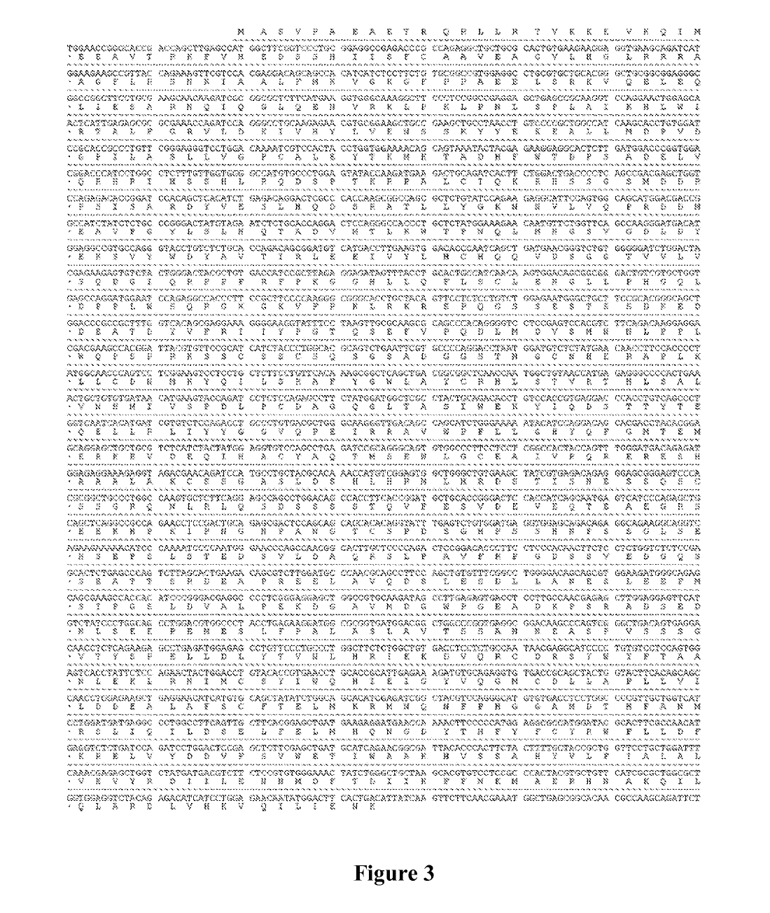

A NuIP includes for example, the nucleic acid (SEQ ID NO:2) and amino acid (SEQ ID NO:1) sequences shown in FIG. 3. For example, provided is a polypeptide comprising less than 1093 amino acids of SEQ ID NO:1. For example, the polypeptide includes at least 10, 15, 20, 25, 30, 35, 40, 45, 50, 100, 200, 300, 400, 500 or 1000 contiguous amino acid residues of SEQ ID NO:1. Optionally, the polypeptides comprise the amino acid sequence CVMDGWPGEADKPSRA (SEQ ID NO:3). Optionally, the NuIP fragment comprises at least 10, 15, 20, 25, 30, 35, 40, 45, 50, 100, 200, 300, 400, 500 or 1000 contiguous amino acid residues of SEQ ID NO:1. Optionally, the fragment includes the amino acid sequence CVMDGWPGEADKPSRA (SEQ ID NO:3). Further, there are a variety of sequences that are disclosed on Genbank, at www.pubmed.gov, for example, the mouse NuIP isoform 1 protein and nucleotide sequences are disclosed at GenBank Accession No. NP_{—}766306 and NM_{—}172718, respectively, and these sequences and others are herein incorporated by reference in their entireties as well as for individual subsequences contained therein. Thus, provided are amino acid sequences comprising an amino acid sequence with at least about 70%, 75%, 80%, 85%, 90%, 95%, 98%, 99% or more percent identity to the amino acid sequence of SEQ ID NO:1. Also provided are nucleic acids comprising a nucleotide sequence with at least about 70%, 75%, 80%, 85%, 86%, 90%, 95%, 98%, 99% or more percent identity to the nucleotide sequence of SEQ ID NO:2 or complement thereof.

As used herein, the term peptide, polypeptide, protein or peptide portion is used broadly herein to mean two or more amino acids linked by a peptide bond. Protein, peptide and polypeptide are also used herein interchangeably to refer to amino acid sequences. The term fragment is used herein to refer to a portion of a full-length polypeptide or protein. It should be recognized that the term polypeptide is not used herein to suggest a particular size or number of amino acids comprising the molecule and that a peptide of the invention can contain up to several amino acid residues or more.

As with all peptides, polypeptides, and proteins, it is understood that substitutions in the amino acid sequence of the NuIP, NuIP analog or fragments of NuIP or NuIP analog can occur that do not alter the nature or function of the peptides, polypeptides, or proteins. Such substitutions include conservative amino acid substitutions and are discussed in greater detail below.

The polypeptides provided herein have a desired function or functions. The polypeptides as described herein selectively bind Nurr1, and may, for example, bind the NLBD of Nurr1. By binding is meant a detectable binding at least about 1.5 times the background of the assay method. For selective or specific binding such a detectable binding can be detected for a given agent but not a control antigen or agent. The polypeptides are tested for their desired activity using the in vitro assays described herein, or by analogous methods, after which their therapeutic, diagnostic or other purification activities are tested according to known testing methods. The polypeptides optionally also have the desired function of activation of Nurr1 function.

The polypeptides described herein can be modified and varied so long as the desired function or functions are maintained. It is understood that one way to define any known variants and derivatives or those that might arise, of the disclosed genes and proteins herein is through defining the variants and derivatives in terms of similarity or identity to specific known sequences. Specifically disclosed are variants of a NuIP having at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the stated sequence. Those of skill in the art readily understand how to determine the identity of two proteins or nucleic acids. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math. 2: 482 (1981), by the identity alignment algorithm of Needleman and Wunsch, J. Mol. Biol. 48: 443 (1970), by the search for similarity method of Pearson and Lipman, Proc. Natl. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by for example the algorithms disclosed in Zuker, Science 244:48-52 (1989); Jaeger et al., Proc. Natl. Acad. Sci. USA 86:7706-7710 (1989); and Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

Fragments, variants, or isoforms of a NuIP are provided. It is understood that these terms include functional fragments and functional variants. For example, fragments can include any portion of the NuIP as long as the fragment binds Nurr1 and, optionally, activates one or more Nurr1 functions.

The variants are produced by making amino acid substitutions, deletions, and insertions, as well as post-translational modifications. Variations in post-translational modifications can include variations in the type or amount of carbohydrate moieties of the protein core or any fragment or derivative thereof. Variations in amino acid sequence may arise naturally as allelic variations (e.g., due to genetic polymorphism), may be produced by human intervention (e.g., by mutagenesis of cloned DNA sequences, such as induced point, deletion, insertion and substitution mutants), or may be produced by environmental intervention (e.g., ultraviolet irradiation). These modifications can result in changes in the amino acid sequence, provide silent mutations, modify a restriction site, or provide other specific mutations.

Protein variants and derivatives can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions

| Amino Acid | Substitutions (others are known in the art) |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

As used herein, modification with reference to a polynucleotide or polypeptide, refers to a naturally-occurring, synthetic, recombinant, or chemical change or difference to the primary, secondary, or tertiary structure of a polynucleotide or polypeptide, as compared to a reference polynucleotide or polypeptide, respectively (e.g., as compared to a wild-type polynucleotide or polypeptide). Modifications include such changes as, for example, deletions, insertions, or substitutions. Polynucleotides and polypeptides having such mutations can be isolated or generated using methods well known in the art.

Nucleic acids that encode the aforementioned peptide sequences, variants and fragments thereof are also disclosed. These sequences include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. A wide variety of expression systems may be used to produce NuIP peptides as well as fragments, isoforms, and variants.

The nucleic acid sequences provided herein are examples of the genus of nucleic acids and are not intended to be limiting. Also provided are expression vectors comprising these nucleic acids, wherein the nucleic acids are operably linked to an expression control sequence. Further provided are cultured cells comprising the expression vectors. Such expression vectors and cultured cells can be used to make the polypeptides of the invention.

There are a variety of molecules disclosed herein that are nucleic acid based, including for example the nucleic acids that encode NuIP or fragments or variants thereof. There are a number of compositions and methods which can be used to deliver nucleic acids to cells, either in vitro or in vivo via, for example expression vectors. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Such methods are well known in the art and readily adaptable for use with the compositions and methods described herein. Further, these methods can be used to target certain diseases and cell populations by using the targeting characteristics of the carrier.

As used herein, plasmid or viral vectors are agents that transport the disclosed nucleic acids into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviral vectors, in general, are described by Verma, I. M., Retroviral vectors for gene transfer. In Microbiology-1985, American Society for Microbiology, pp. 229-32, Washington, (1985), which is incorporated by reference herein. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-1239 (1987); Zhang, BioTechniques 15:868-72 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency after direct, in vivo delivery to airway epithelium, hepatocytes, vascular endothelium, CNS parenchyma and a number of other tissue sites. Other useful systems include, for example, replicating and host-restricted non-replicating vaccinia virus vectors.

The provided polypeptides or nucleic acids can be delivered via virus like particles. Virus like particles (VLPs) consist of viral protein(s) derived from the structural proteins of a virus. Methods for making and using virus like particles are described in, for example, Garcea and Gissmann, Curr. Opin. Biotech. 15:513-7 (2004).

The provided polypeptides can be delivered by subviral dense bodies (DB). Dense bodies transport proteins into target cells by membrane fusion. Methods for making and using DBs are described in, for example, Pepperl-Klindworth et al., Gene Therapy 10(3):278-84 (2003).

The provided polypeptides can be delivered by tegument aggregates. Methods for making and using tegument aggregates are described in International Publication NO. WO 2006/110728.

Also provided are antibodies that specifically bind a NuIP or a fragment or analog thereof. The term antibody is used herein in a broad sense and includes both polyclonal and monoclonal antibodies. Monoclonal antibodies can be made using any procedure that produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 (Burton et al.) and U.S. Pat. No. 6,096,441 (Barbas et al).

Digestion of antibodies to produce fragments thereof, e.g., Fab fragments, can be accomplished using routine techniques known in the art. For instance, digestion can be performed using papain. Examples of papain digestion are described in WO 94/29348 published Dec. 22, 1994 and U.S. Pat. No. 4,342,566 (Theofilopoulos et al.). Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibody fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, Curr. Opin. Biotech. 3:348-54 (1992)).

As used herein, the term antibody or antibodies can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)). Specifically, the homozygous deletion of the antibody heavy chain joining region (J(H)) gene in these chimeric and germ line mutant mice results in complete inhibition of endogenous antibody production, and the successful transfer of the human germ line antibody gene array into such germ line mutant mice results in the production of human antibodies upon antigen challenge.

Antibody humanization techniques generally involve the use of recombinant DNA technology to manipulate the DNA sequence encoding one or more polypeptide chains of an antibody molecule. Accordingly, a humanized form of a non human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. As used throughout, antibody fragments include Fv, Fab, Fab′, or other antigen binding portion of an antibody. Methods for humanizing non human antibodies are well known in the art. For example, humanized antibodies can be generated according to the methods of Winter and co workers (Jones et al., Nature 321:522-5 (1986), Riechmann et al., Nature 332:323-7 (1988), Verhoeyen et al., Science 239:1534-6 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Pharmaceutical compositions comprising one or more of the provided molecules (i.e., antibodies, polypeptides and nucleic acids) herein may include pharmaceutical carriers, thickeners, diluents, buffers, preservatives, surface active agents and the like in addition to the molecule of choice. Pharmaceutical compositions may also include one or more active ingredients such as antimicrobial agents, and the like. The compositions of the present application can be administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution and/or suspension (for example, incorporated into microparticles, liposomes, or cells). These may be targeted to a particular cell type via antibodies, receptors, or receptor ligands. Suitable carriers and their formulations are described in Remington: The Science and Practice of Pharmacy 21^{st} Edition, David B. Troy, ed., Lippincott Williams & Wilkins (2005). Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of composition being administered.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. Optionally, the composition is administered by oral inhalation, nasal inhalation, or intranasal mucosal administration. Adminsitration of the compositions by inhalant can be through the nose or mouth via delivery by spraying or droplet mechanism. For example, in the form of an aerosol.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

The compositions can be formulated to ensure that they cross the blood brain barrier (BBB). They can be formulated, for example, in liposomes. The liposomes may comprise one or more moieties which are selectively transported into specific cells or organs (targeting moieties), thus providing targeted drug delivery. Exemplary targeting moieties include folate, biotin, mannosides, antibodies, surfactant protein A receptor and gp120.

To ensure that agents of the invention cross the BBB, they may also be coupled to a BBB transport vector (see Bickel, et al., Adv. Drug Delivery Reviews, vol. 46, pp. 247-279, 2001). Exemplary transport vectors include cationized albumin or the OX26 monoclonal antibody to the transferrin receptor; these proteins undergo absorptive-mediated and receptor-mediated transcytosis through the BBB, respectively.

Examples of other BBB transport vectors that target receptor-mediated transport systems into the brain include factors such as insulin, insulin-like growth factors (IGF-I, IGF-II), angiotensin II, atrial and brain natriuretic peptide (ANP, BNP), interleukin I (IL-1) and transferrin. Monoclonal antibodies to the receptors which bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized inimunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties such as, for example, glucose; monocarboxylic acids such as, for example, lactic acid; neutral amino acids such as, for example, phenylalanine; amines such as, for example, choline; basic amino acids such as, for example, arginine; nucleosides such as, for example, adenosine; purine bases such as, for example, adenine, and thyroid hormones such as, for example, triiodothyridine. Antibodies to the extracellular domain of nutrient transporters can also be used as transport vectors.

In some cases, the bond linking the agent to the transport vector may be cleaved following transport into the brain in order to liberate the biologically active compound. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like. Generally, the dosage will vary with the age, condition, sex, type of disease and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The provided compositions can be administered in combination with one or more other therapeutic or prophylactic regimens. As used throughout, a therapeutic agent is a compound or composition effective in ameliorating a pathological condition. Illustrative examples of therapeutic agents include, but are not limited to, L-Dopa, or other known agents for treating Parkinson's Disease, anti-inflammatory agents, antibiotics, immunosuppressive agents, and immunoglobulins. Optionally, the provided compositions are administered in combination with a neuroprotective compound. The aforementioned treatments can be used in any combination with the compositions described herein. Thus, for example, the compositions can be administered in combination with a chemotherapeutic agent and radiation. Other combinations can be administered as desired by those of skill in the art. Combinations may be administered either concomitantly (e.g., as an admixture), separately but simultaneously (e.g., via separate intravenous lines into the same subject), or sequentially (e.g., one of the compounds or agents is given first followed by the second). Thus, the term combination is used to refer to either concomitant, simultaneous, or sequential administration of two or more agents.

Methods of screening for agents that modulate the interaction of Nurr1 and NuIP are provided. Such agents may be useful as active ingredients included in pharmaceutical compositions for treating subject suffering from a condition associated with reduced dopaminergic function. The methods include the steps of providing a composition comprising Nurr1 and NuIP, contacting the composition with an agent to be tested, and determining whether the agent to be tested modulates the interaction of Nurr1 and NuIP. The agent can, for example, promote or inhibit the interaction of Nurr1 and NuIP. Optionally, the determining step comprises determining a level of binding of Nurr1 and NuIP. The level of binding of Nurr1 and NuIP can be determined, for example, by selecting an assay from the group consisting of a coimmunoprecipitation assay, a two hybrid assay, and a colocalization assay. The assays are described below and are known in the art, e.g., see Sambrook et al., Molecular Cloning: A Laboratory Manual, 3^{rd} Ed., Cold Spring Harbor Press, Cold Spring Harbor, N.Y. (2001); Dickson, Methods Mol. Biol. 461:735-44 (2008); Nickels, Methods 47(1):53-62 (2009); and Zinchuk et al., Acta Histochem. Cytochem. 40(4):101-11 (2007).

By way of another example, the method includes providing a population of cells, wherein the cells express Nurr1 and NuIP, contacting the cells with an agent to be tested, and determining whether the agent to be tested modulates the interaction of Nurr1 and NuIP. Optionally, the contacting step is in vitro or in vivo. Optionally, the determining step comprises measuring expression of a Nurr1 target gene. Optionally, the Nurr1 target gene is dopamine transporter (DAT). Optionally, the determining step comprises measuring cell number.

The Nurr1 sequence can, for example, be selected from the group consisting of SEQ ID NO:31, 32, 33, 34, and 35. The NuIP sequence can, for example, comprise SEQ ID NO:1.

The provided cells can be made by known methods. For example, the provided cells that express a NuIP and a Nurr1 can be made by delivering to the cell one or more vectors comprising a NuIP and/or a Nurr1 wherein NuIP and Nurr1 are expressed in the cell following delivery of the vector to the cell. The NuIP and Nurr1 can be on the same or different vectors. The cell can be a prokaryotic or a eukaryotic cell.

Agents to be tested include, but are not limited to, small molecules, polypeptides (including antibodies) or nucleic acid molecules.

Assay techniques that can be used to determine levels of expression in a sample are well-known to those of skill in the art. Such assay methods include radioimmunoassays, reverse transcriptase PCR(RT-PCR) assays, immunohistochemistry assays, in situ hybridization assays, competitive-binding assays, Western Blot analyses, ELISA assays and proteomic approaches, two-dimensional gel electrophoresis (2D electrophoresis) and non-gel based approaches such as mass spectrometry or protein interaction profiling. Assays also include, but are not limited to, competitive and non-competitive assay systems using techniques such as radioimmunoassays, enzyme immunoassays (EIA), enzyme linked immunosorbent assay (ELISA), sandwich immunoassays, precipitin reactions, gel diffusion reactions, immunodiffusion assays, agglutination assays, complement-fixation assays, immunoradiometric assays, fluorescent immunoassays, protein A immunoassays, and immunoelectrophoresis assays. For examples of immunoassay methods, see U.S. Pat. No. 4,845,026 and U.S. Pat. No. 5,006,459.

As used herein the terms treatment, treat or treating refer to a method of reducing the effects of a disease or condition or symptom of the disease or condition. Thus in the disclosed method treatment can refer to a 10%, 20%, 30%, 40%, 50%, 60%, 70%, 80%, 90% or 100% reduction in the severity of an established disease or condition or symptom of the disease or condition. For example, the method for treating a protein aggregate disorder is considered to be a treatment if there is at least a 10% reduction in one or more symptoms of the disease in a subject as compared to control. Thus the reduction can be a 10%, 20%, 30%, 40%, 50%, 60%, 70%, 80%, 90%, 100% or any percent reduction in between 10 and 100 as compared to native or control levels. It is understood that treatment does not necessarily refer to a cure or complete ablation of the disease, condition or symptoms of the disease or condition.

As used herein, the terms prevent, preventing and prevention of a disease or disorder refers to an action, for example, of administration of a therapeutic agent, that occurs before a subject begins to suffer from one or more symptoms of the disease or disorder, which inhibits or delays onset of one or more symptoms of the disease or disorder.

As used herein, subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. As used herein, patient or subject may be used interchangeably and can refer to a subject afflicted with a disease or disorder (e.g., Parkinson's Disease). The term patient or subject includes human and veterinary subjects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of aspects have been described. Nevertheless, it will be understood that various modifications may be made. Accordingly, other aspects are within the scope of the following claims.

### EXAMPLES

### General Methods

### Two-Hybrid Screening.

pPC97-NLBD589A was transformed into the yeast strain YRG-2 (Mata ura 3-52his 3-200ade 2-101 lys 2-801 trp 1-901leu 2-3 112gal 4-542gal 80-538LYS2::UASGAL1-TATAGAL1-HIS3 URA3::UASGAL4 17mer(x3)-TATACYC1-lacZ) (Stratagene; La Jolla, Calif.) together with a 13.5 d embryonic mouse ventral mesencephalic cDNA library fused to a Gal4 activation domain pPC86 vector (Chevray and Nathans, Proc. Natl. Acad. Sci. USA 89:5789-93 (1992)). Library screening was performed by using HIS3 and LacZ reporters as described (Stratagene). Candidate interacting clones were purified and retransformed into yeast to confirm the interaction with bait constructs. Confirmed positive clones were identified by sequencing analysis.

### Plasmids.

The different bait constructs were generated by cloning variant Nurr1 cDNA fragments in-frame with the Gal4 DNA-binding domain coding sequence in pPC97 vector. Oligonucleotides listed below were used to amplify the full-length Nurr1 ligand binding domain (NLBD) or the truncated NLBD (NLBD583) from the vector pBSNurr1 by PCR using pfu polymerase: Nurr1-5′ Primer (5′-AGA GTC GAC GGC AGC CAT GCC TTG TGT TCA GGC G-3′) (SEQ ID NO:4), Nurr1-3′ Primer (5′-CTA GGC GGC CGC GGG AGA AGG TCT TAG AAA GGT AA-3′) (SEQ ID NO:5); NLBD-5′ Primer (5′-TAG AGT CGA CCC AGG ATC CCT CTC CCC CCT CAC CT-3′) (SEQ ID NO:6), NLBD-3′ Primer (5′-CTA GGC GGC CGC GGG AGA AGG TCT TAG AAA GGT AA-3′) (SEQ ID NO:5); NLBD Δ583-5′ Primer (5′-TAG AGT CGA CCC AGG ATC CCT CTC CCC CCT CAC CT-3′) (SEQ ID NO:6), NLBD Δ583-3′ Primer (5′-CTA GCG GCC GCT TAT GGT ACC AAG TCT TCC AAT TT-3′) (SEQ ID NO:7).

For each of these oligonucleotides, the 5′ primer introduced a unique SalI site to the 5′ of the coding sequence and the 3′ primer introduced a unique NotI site downstream of the stop codon. The Nurr1 fragment and the pPC97 vector were double digested with SalI and NotI, respectively, and the fragments purified and ligated by T4 DNA ligase. The pPC97Nurr1589A and pPC97NLBD589A plasmids were generated by site-directed mutagenesis.

The pPC86RXR construct was generated by cloning full-length human RXRα cDNA in-frame with the Gal4 DNA activation domain coding sequence in the pPC86 vector. Oligonucleotides listed below were used to amplify the full-length hRXRα from the vector pCMXhRXRα by PCR using pfu polymerase: RXRα-5′ Primer (5′-CTG GGAATT CAC ATG GAC ACC AAA CAT TTC-3′) (SEQ ID NO:8) and RXRα-3′ Primer (5′-CTAAGC GGC CGC CTAAGT CAT TTG GTG CGG-3′) (SEQ ID NO:9). Amplified PCR fragments were inserted into the pPC86 vector. Oligonucleotides used in all studies were synthesized by Integrated DNA Technologies. All sequences were verified by sequencing (Integrated DNA Technologies; Coralville, Iowa).

The pHSV-green fluorescent protein (GFP)/Nurr1 construct contains the coding sequences for both humanized Renilla reniformis GFP and NUrr1 under the control of separate promoters in the plasmid HSVPrPucλ₃CMV and was generated as described previously (Luo and Federoff, Ann. N.Y. Acad. Sci. 991:350-3 (2003)). A reporter construct containing three copies of NBRE upstream of a minimal promoter driving luciferase was generated as described previously (Luo and Federoff, Ann N.Y. Acad. Sci. 991:350-3 (2003)). Reporter constructs containing −3 kb, −6 kb, or −9 kb for the rat TH promoter were constructed. Briefly, the fragment of endogenous rat TH promoter of −9 kb, −6 kb, or −3 kb was cloned into the HSVlacZ vector upstream of a LacZ reporter gene. Flag-tagged full-length Nurr1 expression vector was generated by cloning a Nurr1 PCR fragment in frame fused to the 3′ terminal of a Flag sequence into a pcDNA3 vector (Clontech; Mountain View, Calif.).

The full-length NuIP gene was cloned by reverse transcriptase (RT)-PCR using cDNA library from embryonic 13.5 (E13.5) mouse ventral midbrain as template and the following oligos as primers: NuIP Full Length-5′ Primer (5′-TAG AGT CGA CGG AAC CGG GCA CCG ACC AGC TTG AGC CA-3′) (SEQ ID NO:10) and NuIP Full Length-3′ Primer (5′-CTA GTC TAG ACT TGT TCT CAA TTA GAA TCT G-3′) (SEQ ID NO:11). The amplified PCR fragment was inserted into the SalI and XbaI sites of the HSVPrPuc expression vector, and the sequence was confirmed by sequencing analysis (Integrated DNA Technologies). VP16NuIP was generated by cloning a PCR fragment of NuIP into the SalI and XbaI sites of pVP16 vector (Clontech) in-frame with the VP16 activation domain using a different 5′ primer: VP16 NuIP-5′ Primer (5′-TAG AGT CGA CGG CAC CGA CCA GCT TCA GCC A-3′) (SEQ ID NO:12). NuIPV5 was generated by cloning a NuIP PCR fragment in-frame fused to the 5′ terminal of a V5 sequence into a modified pVP16 vector in which the VP16 activation domain sequence is deleted.

The Gal4H1 construct was cloned by inserting a PCR fragment containing the helix 1 (H1) sequence of NLBD into the pPM vector (Clontech) in-frame with the Gal4 DB sequence. The VP16H3-12 construct was cloned by inserting a PCR fragment containing the helix 3-12 sequence of NLBD into the pVP16 vector (Clontech) in-frame with the VP16 activation domain sequence. The following primers were used to generate the PCR fragments: H1-5′ Primer (5′-AGA GTC GAC CGA AGA GCC CAC AGG ATC CCT CT-3′) (SEQ ID NO:13), H1-3′ Primer (5′-CTA GTC TAG AAT CTC CAC TCA TCT GAT AGT CAG G-3′) (SEQ ID NO:14); H3-12-5′ Primer (5′-TAG AGT CGA CAT GAT ACC CAA CAT ATC CAG CAG-3′) (SEQ ID NO:15), H3-12-3′ Primer (5′-CTA GTC TAG AGG GAG AAG GTC TTA GAA AGG TAA-3′) (SEQ ID NO:16).

Oligonucleotides used in all studies were synthesized by Integrated DNA Technologies. All constructions were verified by sequencing (Integrated DNA Technologies).

### Transfections and Reporter Assay.

MN9D cells were plated at 1.5×10⁵ cells/well in 24-well plates coated with PEI 24 hours before transfection. Cells were washed with Optimem (Invitrogen; Carlsbad, Calif.) and incubated with DNA and 1.5 μl of Lipofectamine 2000 (Invitrogen) in Optimem for 8 hours. Each well was transfected with 100 ng of individual reporter construct, 500 ng of pHSV-GFP/Nurr1, and 250 ng of HSVNuIP or pBS as carrier DNA. 50 ng of pRL-Null reference plasmid containing the Renilla luciferase gene was used as an internal control and for normalization of transfection efficiency. After an 8 hour incubation, the transfection reagents were removed and replaced with DMEM with 10% FBS. Luciferase and Renilla luciferase activities were assayed 24 hours later using the Dual-Luciferase reporter assay system according to the manufacture's instructions (Promega; Madison, Wis.). Luciferase activities were normalized to the Renilla luciferase activity. Each assay was performed a minimum of three times for each condition and average values±SD are shown for each sample.

### Mammalian Two-Hybrid Assay.

The interaction between Nurr1 and NuIP was examined using the mammalian two-hybrid assay. MN9Dcells were cotransfected with pGal4NLBD or pGal4NLBD583 and pVP16NuIP constructs along with a reporter gene driven by five copies of the Gal4-binding sites. The cells were subsequently harvested and analyzed as described above for reporter activity.

### Nurr1 LBD Assembly Assay.

The assembly of H1 and H3-12 domain of NLBD was assessed by using the NLBD assembly assay (Wang et al., Nature 423:555-60 (2003)). HEK293 cells were cotransfected with the reporter construct, Gal4H1, and VP16H3-12 together with HSVX NuIP or pBS as carrier DNA. The cells were then harvested and analyzed for luciferase activity and normalized to reference Renilla luciferase activity. Each condition was measured with triplicate samples, and each experiment was repeated at least three times with similar results.

### Development and Characterization of NuIP Specific Antibody.

An NuIP specific peptide (CVMDGWPGEADKPSRA) (SEQ ID NO:3) was used to immunize rabbits (Affinity Bioreagents; Rockford, Ill.). After three immunization boosts, immune sera containing IgG were purified and tested for specificity for the NuIP protein. Briefly, a clonal MN9D cell line was transfected with either a control plasmid (HSVlacZ) or a NuIP-expressing vector (HSVNuIP). Twenty four hours after transfection, cell lysates were prepared using modified radioimmunoprecipitation assay (RIPA) buffer. Total protein (20 μg) from each condition was separated on an 8% SDS-PAGE gel and transferred to a polyvinylidene fluoride (PVDF) membrane for Western blot analysis. The membrane was incubated with 1 μg/ml NuIP antibody for 1 hour at room temperature, followed by a 45 minute incubation of secondary HRP-conjugated anti-rabbit antibody (Jackson ImmunoResearch Laboratories; West Grove, Pa.). For peptide preabsorption, the NuIP antibody solution was preincubated with an excess of the NuIP specific peptide (10 μg/ml) for 30 minutes at room temperature before the incubation with membrane.

### Communoprecipitation.

MN9D cells were cotransfected with a NuIP-V5 expression vector and a Flag-Nurr1 expression vector. Twenty four hours after transfection, nuclear extracts were prepared by NE-PER nuclear extraction reagent (Pierce Chemical; Rockford, Ill.). Nuclear lysate was precleared with Protein A beads and was incubated with rabbit polyclonal anti-V5 antibody (Novas Biologicals; Littleton, Colo.) in a modified RIPA buffer or with RIPA buffer only (no antibody) as a control. Protein A beads were incubated with the antibody/buffer:lysates for 1 hour, washed five times with RIPA buffer, and bound proteins eluted by boiling in Laemmli sample buffer. Proteins were subjected to denaturing PAGE (6% SDS-PAGE), transferred to PVDF membrane, and probed with a monoclonal V5 antibody (Invitrogen) or a monoclonal Flag antibody (Sigma-Aldrich; St. Louis, Mo.). Communoprecipitation of endogenous Nurr1-NuIP complexes was performed by incubating 120 μg of mouse SN lysates with rabbit polyclonal anti-Nurr1 antibody (Novas Biologicals) in PBS or with PBS only (no antibody) as a control. Use of protein A beads, their incubation, and washing procedures were performed as described above. Proteins were separated by SDS-PAGE and blotted with rabbit polyclonal anti-NuIP antibody (described above).

### Reverse Transcription-Polymerase Chain Reaction (RT-PCR).

Total RNA from cells was extracted by Trizol reagent (Invitrogen, Carlsbad, Calif.). Total RNA (2 μg) was treated with RQ-1 RNase-free DNase I and reverse transcribed into cDNA using random hexamers by AMV reverse transcriptase as recommended by manufacturer's protocol (Roche Diagnostics; Indianapolis, Ind.). Synthesized cDNAs were then subject to PCR amplification using the following primers to detect different transcripts: NuIP: forward primer (5′-TTGAAGTGGACACCCAATCAGC-3′) (SEQ ID NO:17), reverse primer (5′-CAGGTCTGGAGACACGATCATGTG-3′) (SEQ ID NO:18); NuIPa: forward primer (5′-CTCTGGCTTCCCACAGTCTCCC-3′) (SEQ ID NO:19), reverse primer (5′-CAGGTCTGGAGACACGATCATGTG-3′) (SEQ ID NO:20); NuIPb: forward primer (5′-CTCTGGCTTCCCACAGTCTCCC-3′) (SEQ ID NO:21), reverse primer (5′-GCTTCTTGCAGACAACAGCAGG-3′) (SEQ ID NO:22); NuIPc: forward primer (5′-TTGAAGTGGACACCCAATCAGC-3′) (SEQ ID NO:23), reverse primer (5′-GCTTCTTGCAGACAACAGCAGG-3′) (SEQ ID NO:24); Nurr1: forward primer (5′-ATTCCAATCCGG CAATGACC-3′) (SEQ ID NO:25), reverse primer (5′-TTGCAACCTGTGCAAGACCAC-3′) (SEQ ID NO:26).

Supression of NuIP Expression by siRNA.

MN9D cells were maintained at 37° C. and 5% CO₂ in DMEM with L-glutamine and 4500 mg/L glucose (Sigma-Aldrich, St. Louis, Mo.) and 3.7 g of sodium bicarbonate per litter added, pH 7.4, and 10% fetal bovine serum. Inducible cell lines harboring NuIP short hairpin RNA (shRNA) constructs were prepared in MN9D cells by stable transfection with pcDNA6/TR vector, selection and expansion in the presence of 5 μg/ml blasticidin for 2 weeks, and screening for induction of expression of a vector-based lacZ reporter gene (pcDNA4/TO/lacZ) by β-galactosidase assay (Invitrogen). The clone expressing the highest level of functional tetracycline repressor was then used to establish inducible NUIP RNA interference (RNAi) cell lines. Short hairpin DNA sequences against the coding sequence of NuIP mRNA were designed using two different approaches (Reynolds et al., Nat. Biotechnol. 22:326-30 (2004); Huesken et al., Nat. Biotechnol. 23:995-1001 (2005)) and cloned into pSUPERIOR neo+GFP vector (Oligoengine; Seatttle, Wash.) following manufacturer's instructions. Multiple hairpin constructs were screened for effective knockdown of NuIP. Of these different constructs two were used in this study and are described as follows: RNAi#1, 5′-GTACCAGATCCTCTCCAGA-3′ (SEQ ID NO:27); and RNAi#2, 5′-CCCGGGACCTCGTGCATAA-3′ (SEQ ID NO:28).

To check NuIP mRNA knockdown, total RNA was extracted using the RNeasy kit (QIAGEN; Valencia, Calif.) from appropriate cell samples 24 hours after induction of shRNA expression, followed by cDNA synthesis using the high capacity cDNA Archive kit (Applied Biosystems; Foster City, Calif.) and quantitative RT-PCR (qRT-PCR). The probes for qRT-PCR of NuIP (Taqman Gene Expression Assays Mm00554850 ml; Applied Biosystems) amplify 140 nt around exon boundary 21-22 with the assay location at 2845 of the cDNA (NM_{—}172718.1). NuIP mRNA levels were normalized to 18S rRNA using probes purchased from Applied Biosystems. qRT-PCR was performed in triplicate.

### Cell Number Counting.

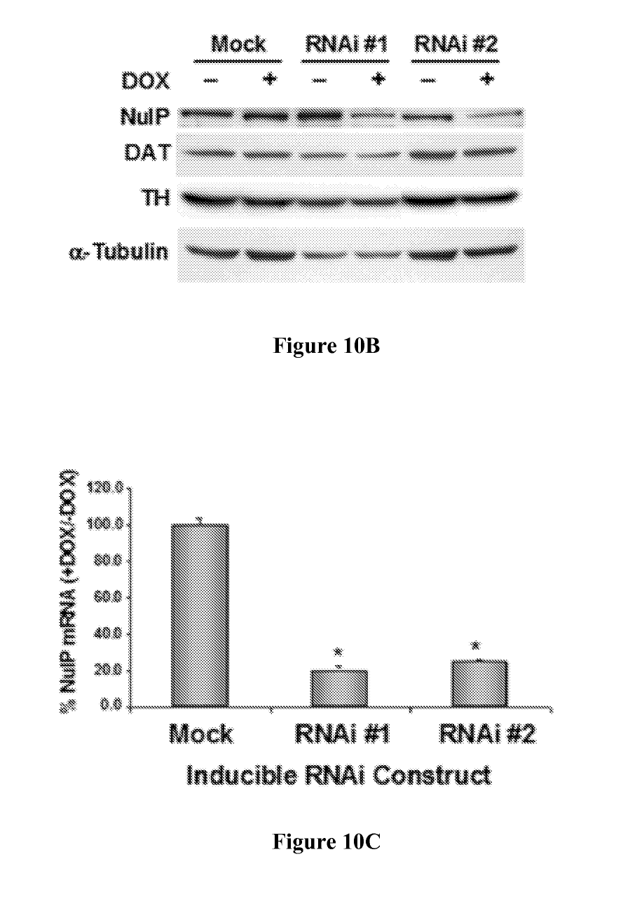

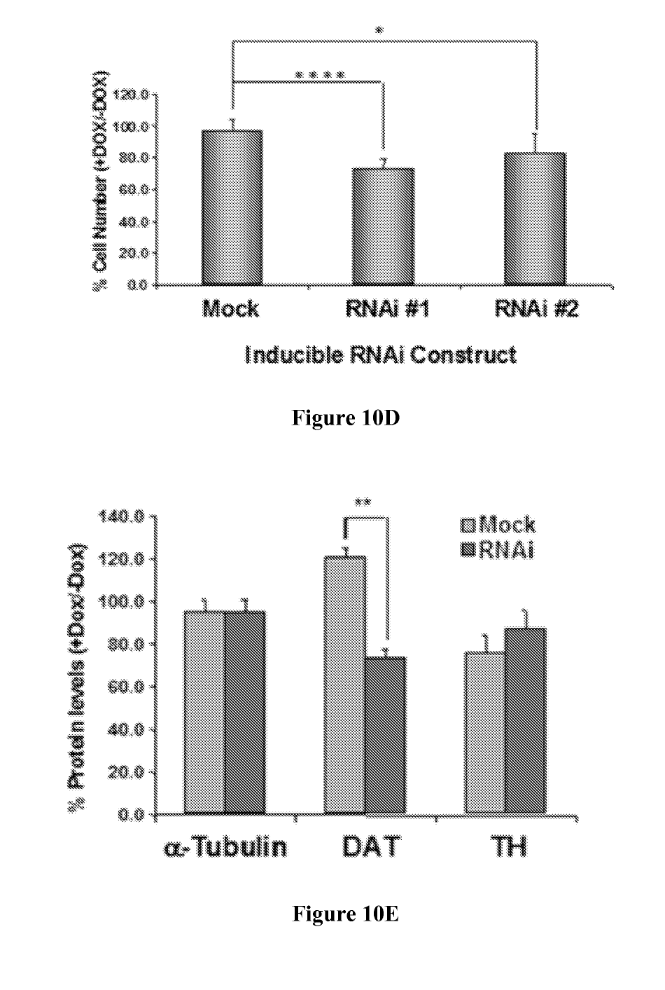

Three days after induction of shRNA expression in tet repressor-expressing MN9D cells in 12-well plates, the cells were trypsinized and counted using a hemocytometer. The percentage of the number of the cells with shRNA expressed (treated with doxycycline: +DOX) versus the cells without shRNA expressed (untreated: −DOX) was calculated. The data shown in FIG. 10 represented an average of seven independent assays each performed in duplicate.

### Western Blotting and Immunocytochemistry.

To measure suppression of NuIP protein by siRNA, tet repressor-expressing MN9D cells were plated 1 day before transfection in polyethyleneimine-coated dishes. The cells were then transfected with either null pSUPER10R neo+GFP vector (mock) or constructs containing RNAi#1 and RNAi#2 sequences, using Transfectamine 2000 diluted in Opti-MEM (Invitrogen), according to the manufacturer's protocol. Doxycycline (DOX) was added to a final concentration of 2 μg/ml 24 hours after transfection to induce shRNA expression. The cells were collected 72 hours after induction for Western blot analysis of various proteins. Total protein (20 μg) from each condition was separated on an 8% SDS-PAGE gel and transferred to a PDVF membrane for Western blot analysis. The blots were then probed by various antibodies: anti-NuIP Ab (described above), anti-DAT (Novus Biologicals), anti-TH (Millipore; Billerica, Mass.) and anti-α Tubulin (Santa Cruz Biochemicals; Santa Cruz, Calif.).

For NuIP and TH double-labeling immunocytochemistry, adult male C57BL/6J mice (2-3 months) were anesthetized and perfused with 4% paraformaldehyde (PEA), pH 7.2. Brains were removed and postfixed in 4% PFA for 24 hours, followed by immersion in 20% sucrose in 0.1 M phosphate buffer, pH 7.2, for 3 days and immersion in 30% sucrose for 1 week. The brains were sectioned at 30 μm on a microtome and mounted on gelatin-coated slides. Immunohistochemistry was performed to identify TH-immunoreactive (IR) and NuIP-IR cells in the SN. Briefly, sections were blocked in PBS containing 10% normal goat serum (Jackson ImmunoResearch Laboratories), 2% BSA, and 0.3% Triton X-100 for 3 hours, followed by incubation in primary antibody (mouse anti-TH, 1:500; rabbit anti-NuIP, 1:1000; INCSTAR) overnight at 4° C. After washes in PBS, sections were incubated with Alexa Fluor 594 conjugated goat anti-rabbit secondary antibody (1:1000; Invitrogen) and Alexa Fluor green 488 conjugated goat anti-mouse secondary antibody (1:1000; Invitrogen) for 1 hour at 25° C. After additional washes (3×) with PBS, the slides were examined under a confocal microscope (Olympus BX50WI; Olympus FluoView) (Olympus America, Inc.; Center Valley, Pa.).

### Example 1

### Identification of Nurr1-Interacting Protein (NuIP)

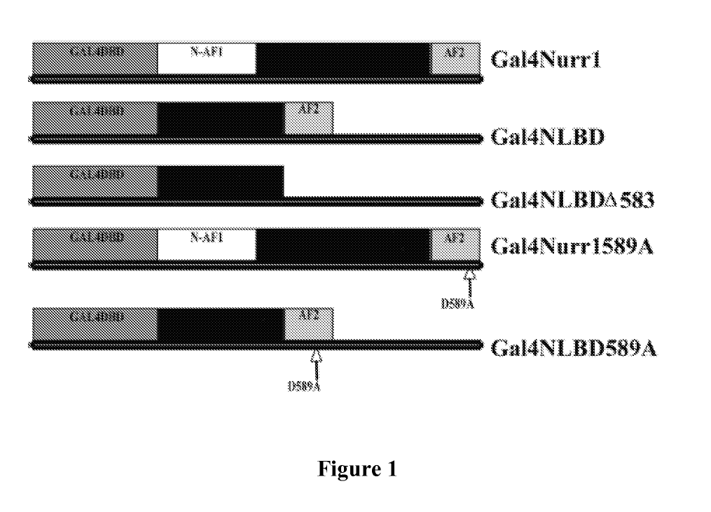

In order to identify potential Nurr1 interacting proteins, a yeast two-hybrid system was used. A mouse E13.5 ventral midbrain library was constructed in the yeast two-hybrid vector PC86. A set of Gal4-Nurr1 fusion constructs was prepared (FIG. 1), which included the full-length Nurr1 (GalNurr1) (SEQ ID NO:31), the entire Nurr1 LBD (GalNLBD) (SEQ ID NO:32), Nurr1 LBD deleted of AF2 (Gal4NLBDp583) (SEQ ID NO:33), and a full-length Nurr1LBD carrying a transcription inactivating mutation in AF2 (Gal4NLBD589A) (SEQ ID NO:34). Attributable to the intrinsic activity of the AF2 domain within the Nurr1LBDdomain (Table 2), the library was screened with a construct containing the inactivating mutation Gal4NLBD589A (SEQ ID NO:35). The selection produced several clones capable of interaction, only one of which was subsequently confirmed on replication. This clone, provisionally termed NuIP, contained a partial opening reading frame corresponding to a gene of unknown function.

TABLE 2
Activity of reporter genes with various Nurr1 bait constructs.

| | With pPC89 | | With pPC89RXR | |
|---|---|---|---|---|
| Constructs | -His | X-gal | -His | X-gal |
| Gal4Nurr1 | Growth | Blue | Growth | Blue |
| Gal4NLBD | Growth | Blue | Growth | Blue |
| Gal4NLBDΔ583 | No Growth | White | No Growth | White |
| Gal4Nurr1589A | Growth | Blue | Growth | Blue |
| Gal4NLBD589A | No Growth | White | Growth | Blue |

To confirm the specificity of the interaction and determine whether the interaction interface with Nurr1LBD was similar to that of RXRα, additional studies in yeast were undertaken. As shown in Table 3, pPC86-NuIP, unlike RXRα, interacted with the LBD of Nurr1 devoid of its AF2 domain. Thus, the protein interface in the NLBD that interacts with NuIP is different from that of RXRα.

TABLE 3
Identification of a positive interactor (X) for Nurr1 protein

| | Results | |
|---|---|---|
| Yeast Transformation | -Leu/-Trp/-His | X-gal staining |
| Gal4NLBD589A + pPC86 | No Growth | White |
| Gal4NLBD589A + pPC86-NuIP | Growth | Blue |
| Gal4NLBDΔ583 + pPC86-NuIP | Growth | Blue |
| Gal4NLBDΔ583 + pPC86-RXR | No Growth | White |

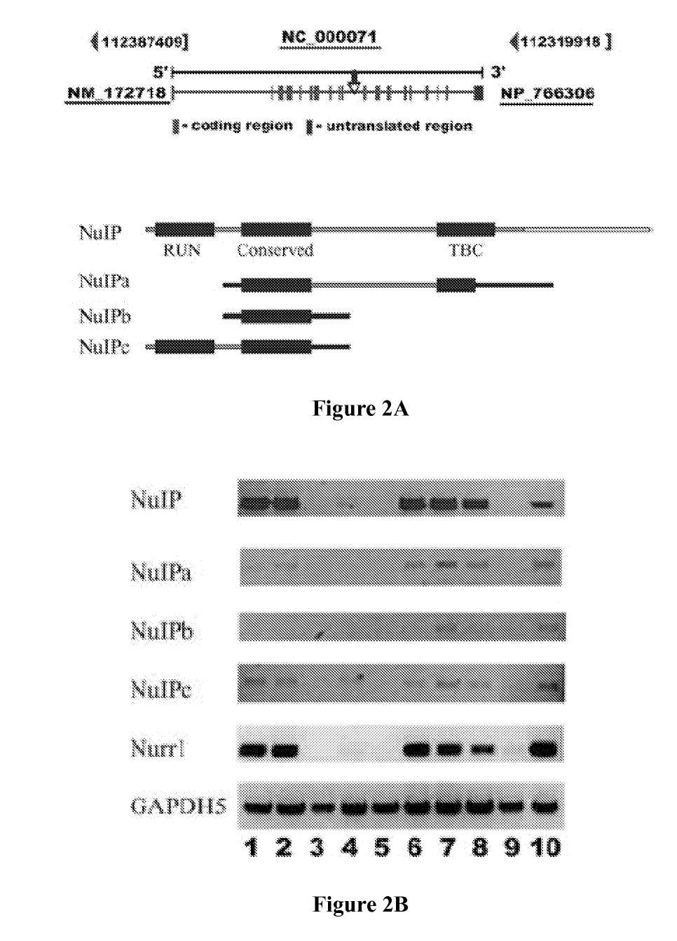

In silico analysis (Vector NTI; Invitrogen) of the partial NuIP ORF indicated that it belongs to a family of transcripts that is expressed in both human and mouse. Reconstruction of the transcription unit from disparate DNA sequence data suggests a single gene with four alternatively processed transcripts (FIG. 2A). To confirm the existence of each transcript, specific PCR primers were designed, and RNA from different adult tissues from the mouse was examined by RT-PCR. As shown in FIG. 2B, all transcripts were expressed, but the patterns of expression indicate preferential abundance of some isoforms in some tissues and not others. There was general concordance of expression of full-length NuIP, NuIPa, and NuIPc in midbrain cortex, striatum, cerebellum, pons/medulla, and embryonic ventral mesencephalon. This overall pattern mirrors the expression of Nurr1. The expression of NuIPb was observed only in cerebellum and embryonic ventral mesencephalon. No expression of any NuIP isoform was detected in the spleen or the heart, but weak expression patterns of full-length NuIP and NuIPc, as well as Nurr1, were observed in kidney. Based on the analysis of mRNA products, a full-length cDNA was cloned encoding the longest isoform of NuIP from mouse prenatal midbrain (E13.5). This gene has a predicted ORF spanning 3.3 kb and encodes a protein product of 1093 amino acids (aa) (FIG. 3). Importantly, the high degree of correlation between Nurr1 and NuIP mRNA expression (FIG. 2B) in multiple brain regions provides a biological context for their potential to interact at the protein level in these tissues.

### Example 2

### Nurr1 and NuIP Functionally Interact in Mammalian Cells

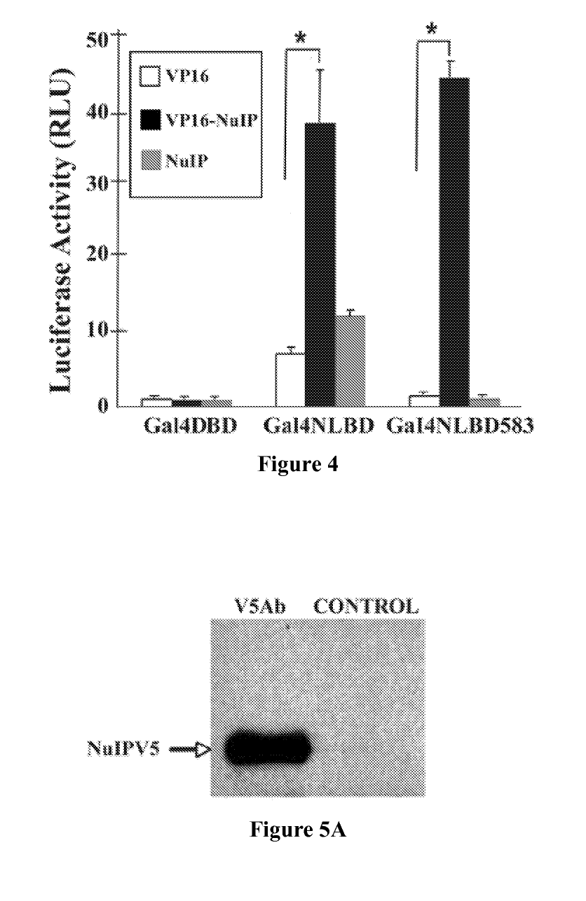

To examine the functional relationship of Nurr1 and NuIP, a mammalian two-hybrid interaction approach was used. The strong transcriptional activator HSV VP-16 was fused to full-length NuIP and co-transfected with the Gal4 DB, Gal4NLBD, or GAL4NLBDA583, together with a Gal4 responsive reporter into the MN9D dopaminergic cell line (Hermanson et al., Exp. Cell. Res. 288:324-34 (2003)). As shown in FIG. 4, significant transactivation was observed with both NLBD constructs, suggesting a functional interaction in this mammalian dopaminergic cell line. In contrast to the reported RXR interaction with Nurr1, NuIP interacts with an NLBD construct lacking the AF2 domain (NLBD583) (FIG. 4), an observation in agreement with the yeast interaction data.

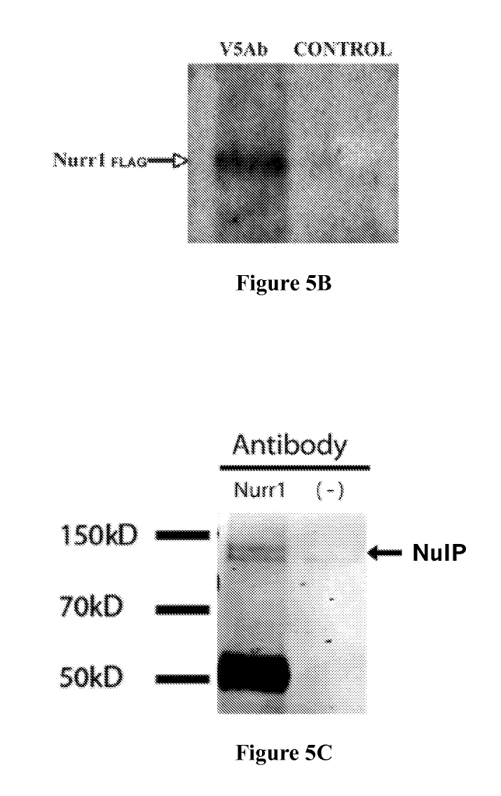

Evidence for interaction of NuIP and NLBD in mammalian cells was also demonstrated by coimmunoprecipitation studies. In these experiments, MN9D cells were transfected with full-length and epitope-tagged constructs of Nurr1 and NuIP and then subjected to epitope specific immunoprecipitation followed by SDS-PAGE and Western blotting. As shown in FIG. 5, immunoprecipitation of NuIP-V5 resulted in the coprecipitation of protein Nurr1-FLAG. To test whether endogenous Nurr1 and NuIP protein interact with each other in SN tissue, rich in dopaminergic neurons, coimmunoprecipitation in tissue lysates was also performed. The result shows that endogenous NuIP protein is coimmunoprecipitated by a rabbit polyclonal anti-Nurr1 antibody (FIG. 5C).

### Example 3

### NuIP Augments the Transcriptional Activity of Nurr1

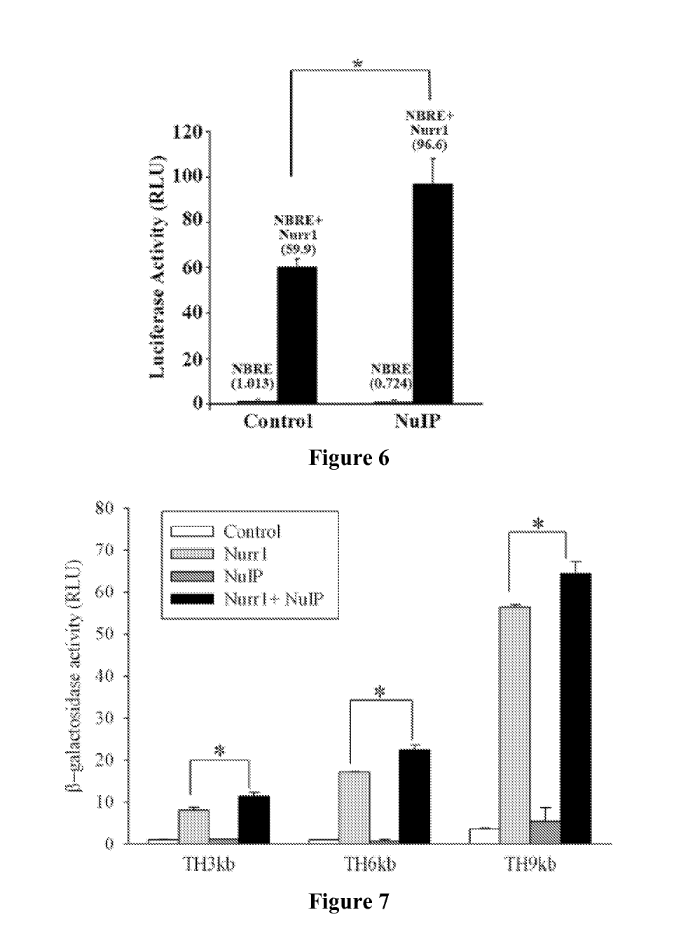

The transactivation function of Nurr1 has been shown using a hybrid promoter containing nerve growth factor inducible-B response element (NBRE) cis-elements (Wilson et al., Science 252:1296-1300 (1991)) and, more recently, using the promoter driving expression of TH (Iwawaki et al., Biochem. Biophys. Res. Commun. 274:590-5 (2000)). To examine whether NuIP can modify the functional properties of Nurr1, full-length constructs of each were prepared in mammalian expression vectors and then used in standard promoter reporter transfection studies in MN9D cells. Transfection of the NBRE-reporter construct alone produced a small but detectable amount of gene expression (FIG. 6). Cotransfection with full-length Nurr1 resulted in a marked transcriptional enhancement. In contrast, cotransfection of full-length protein NuIP along with the reporter construct alone produced no change in reporter gene expression. Interestingly, cotransfection of Nurr1 and NuIP resulted in significantly increased gene expression. This was not attributable to an effect on the abundance of Nurr1 in these transfected cells as assessed by Western blotting. A similar set of experiments was undertaken in MN9D cells, testing whether the same set of effector constructs, Nurr1 and NuIP, would modify the transcriptional activity of the TH promoter. As shown in FIG. 7, NuIP consistently increased the transcriptional activity of Nurr1 on all three TH constructs. Together, these results indicate that NuIP augments the transcriptional activity of Nurr1 .

### Example 4

### NuIP Protein Promotes the Assembly of Helical Domains 1 and 3-12 of the Nurr1 LBD

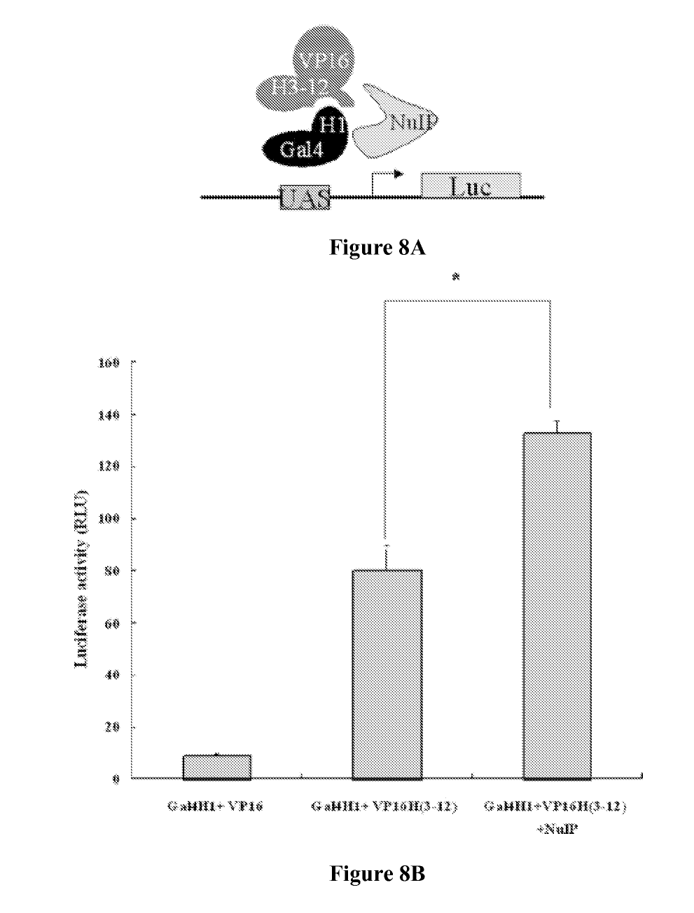

To explore the possible mechanism whereby NuIP potentiates the transcriptional activity of Nurr1, it was tested whether NuIP can promote the assembly of H1 domain and H3-12 domains of the NLBD, which have been shown to correlate with the transcriptional activity of NLBD (Wang et al., Nature 423:555-60 (2003)). The assay is designed to examine whether NuIP can serve as a scaffold for the binding of the separate helical domains and promote their assembly into a transcriptionally competent complex (FIG. 8A). As shown in FIG. 8B, the H1 domain and H3-12 domains interact with each other in transfected HEK293 cells, as demonstrated by increased activity of the reporter gene. Importantly, when cotransfected with NuIP, this interaction is significantly augmented (p<0.001).

### Example 5

### NuIP Protein is Expressed in Nurr1 Containing SN Dopaminergic Neurons

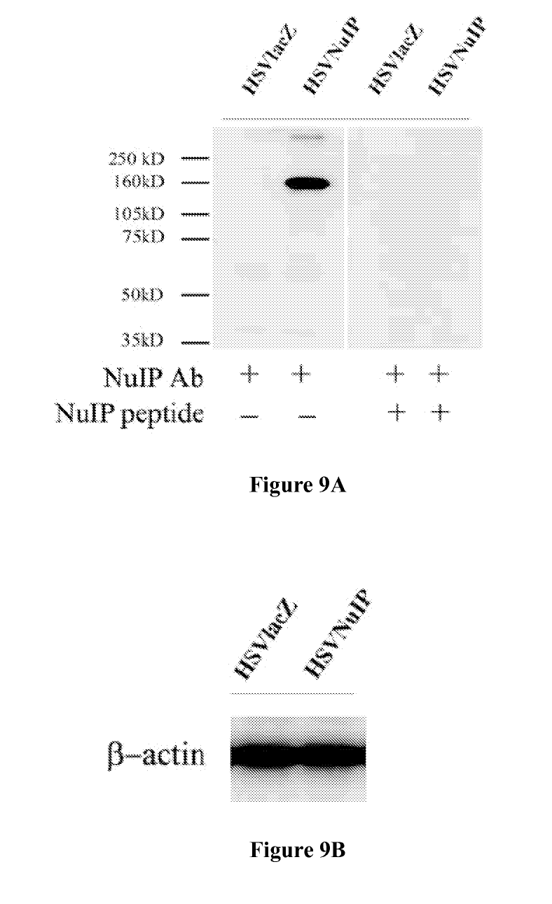

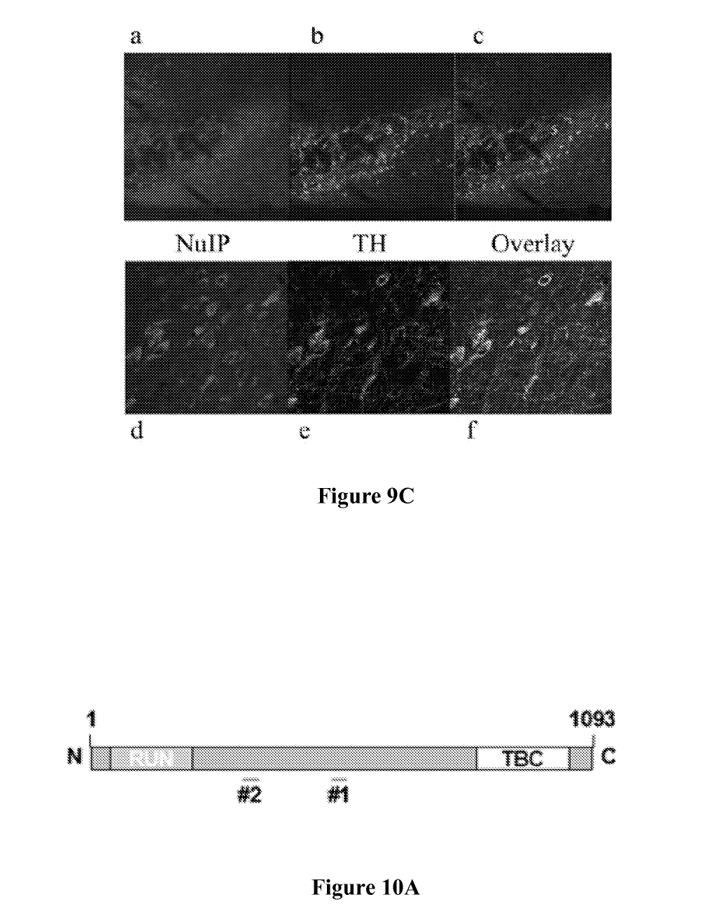

To further test whether NuIP is expressed in Nurr1 containing cells, specifically SN dopaminergic neurons, polyclonal antisera was raised to a unique NuIP peptide. The specificity of the NuIP antibody was confirmed by Western blot and peptide preabsorption. This antibody reveals a band that is the approximate predicted molecular weight of the NuIP protein (150 kDa) only in the cell lysates that are transfected with a NuIP expressing construct (HSVNuIP) (FIG. 9A) and not in lysates from cells transfected with a control plasmid (HSVlacZ). When the antibody was pre-incubated with an NuIP specific peptide, the NuIP signal was lost, indicating that the antibody specifically recognizes NuIP. Blotting with a β-actin antibody confirmed equivalent protein loading (FIG. 9B). Immunohistochemical analysis of adult brain tissue with the anti-NuIP antibody discloses expression in the ventral midbrain dopaminergic group and extensive colocalization with TH immunoreactivity (FIG. 9C), which is readily apparent when the labeling for NuIP and TH are overlaid (FIG. 9C, sections a and f). This result shows that Nurr1 and NuIP are coexpressed within the dopaminergic cells in the adult mouse SN.

### Example 6

### Suppression of Endogenous NuIP Function Results in Decreased Cell Proliferation and Expression of Nurr1 Target Gene

To investigate the outcome of NuIP loss of function, inducible siRNA was used to suppress the expression of NuIP protein. As shown in FIG. 10A, two siRNA sequences target regions between the RUN and TBC domain that are specific for NuIP gene. Induction of siRNA expression by doxycycline (DOX) led to a decrease in NuIP expression in MN9D cells both on the protein (FIG. 10B) and mRNA (FIG. 10C) levels, whereas the mock construct did not alter NuIP expression (FIGS. 10B and 10C). Because Nurr1 has been demonstrated to be important in promoting a dopaminergic phenotype in cells and in regulating several downstream targets that are involved in dopamine synthesis and recycle, it was tested whether suppression of NuIP function would affect the expression level of these Nurr1 target genes. The data show that suppression of NuIP function led to a reduction of DAT protein, a known Nurr1 target (FIGS. 10B and 10E). To determine whether decreased NuIP function would alter dopaminergic MN9D cell phenotype, cell number was examined in siRNA induced and mock transfected cells. The data, shown in FIG. 10D, reveal that reduction in NuIP function results in diminished cell numbers 72 hours after siRNA induction.

1. A method of promoting the activity of Nurr1 in a cell comprising contacting the cell with NuIP or an analog or fragment thereof.

2. The method of claim 1, wherein the promoted activity is expression of a Nurr1 target gene.

3. The method of claim 2, wherein the gene is tyrosine hydroxylase or a nerve growth factor inducible gene.

4. (canceled)

5. The method of claim 1, wherein the cell is a dopaminergic neuron.

6. The method of claim 5, wherein the promoted activity is an increase in cell proliferation.

7. A method of treating or preventing a condition associated with reduced dopaminergic function in a subject, comprising administering to the subject NuIP or an analog or fragment thereof.

8. The method of claim 7, wherein the condition associated with reduced dopaminergic function is Parkinson's Disease, attention deficit disorder, dementia with lewy body or diffuse lewy body with Parkinson's Disease.

9. (canceled)

10. The method of claim 7, further comprising selecting a subject with or at risk of developing Parkinson's Disease.

11. (canceled)

12. The method of claim 1, wherein the NuIP analog is an agonistic antibody to Nurr1.

13. The method of claim 1, wherein the NuIP analog is a small molecule.

14. The method of claim 1, wherein the NuIP fragment comprises the amino acid sequence CVMDGWPGEADKPSRA (SEQ ID NO:3).

15. A method of inhibiting the activity of Nurr1 in a cell comprising contacting the cell with a NuIP siRNA molecule.

16. (canceled)

17. The method of claim 15, wherein the NuIP siRNA molecule targets SEQ ID NO:27 or SEQ ID NO:28.

18. (canceled)

19. A polypeptide comprising less than 1093 amino acids and comprising the amino acid sequence CVMDGWPGEADKPSRA (SEQ ID NO:3).

20. The polypeptide of claim 19, wherein the polypeptide is CVMDGWPGEADKPSRA (SEQ ID NO:3).

21. An antibody that specifically binds the polypeptide of claim 19.

22. A nucleic acid that encodes the polypeptide of claim 19.

23. A pharmaceutical composition comprising the polypeptide of claim 19 and a pharmaceutical carrier.

24. A method of screening for agents that modulate the interaction of Nurr1 and NuIP comprising:
(a) providing a composition comprising Nurr1 and NuIP;
(b) contacting the composition with an agent to be tested; and
(c) determining whether the agent to be tested modulates the interaction of Nurr1 and NuIP.

25. The method of claim 24, wherein the agent promotes the interaction of Nurr1 and NuIP.

26. The method of claim 24, wherein the agent inhibits the interaction of Nurr1 and NuIP.

27. The method of claim 24, wherein the determining step comprises determining a level of binding of Nurr1 and NuIP.

28. The method of claim 27, wherein determining the level of binding of Nurr1 and NuIP involves selecting an assay from the group consisting of a coimmunoprecipitation assay, a two hybrid assay, and a colocalization assay.

29. The method of claim 24, wherein the Nurr1 sequence is selected from the group consisting of SEQ ID NO:31, 32, 33, 34, and 35.

30. The method of claim 24, wherein the NuIP comprises SEQ ID NO:1.

31. (canceled)

32. A method of screening for agents that modulate the interaction of Nurr1 and NuIP comprising:
(a) providing a population of cells, wherein the cells express Nurr1 and NuIP;
(b) contacting the cells with an agent to be tested; and
(c) determining whether the agent to be tested modulates the interaction of Nurr1 and NuIP.

33. (canceled)

34. (canceled)

35. The method of claim 32, wherein the determining step comprises measuring expression of a Nurr1 target gene.

36. The method of claim 35, wherein the Nurr1 target gene is dopamine transporter (DAT).

37. The method of claim 32, wherein the determining step comprises measuring cell number.

38. The method of claim 32, wherein the Nurr1 sequence is selected from the group consisting of SEQ ID NO:31, 32, 33, 34, and 35.

39. The method of claim 32, wherein the NuIP comprises SEQ ID NO:1.

40. (canceled)

41. The method of claim 7, wherein the NuIP analog is an agonistic antibody to Nurr1 .

42. The method of claim 7, wherein the NuIP analog is a small molecule.

43. The method of claim 7, wherein the NuIP fragment comprises the amino acid sequence CVMDGWPGEADKPSRA (SEQ ID NO:3).


SEQUENCE LISTING (35 sequences)

SEQ ID NO: 1 - protein, 1093 aa, Mus musculus
MASVPAEAETRQRLLRTVKKEVKQIMEEAVTRKFVHEDSSHIISFCAAVEACVLHGLRRRAAGFLRSNKIAALFMKVGKGFPPAEELSRKVQELEQLIESARNQIQGLQENVRKLPKLPNLSPLAIKHLWIRTALFERVLDKIVHYLVENSSKYYEKEALLMDPVDGPILASLLVGPCALEYTKMKTADHFWTDPSADELVQRHRIHSSHLRQDSPTKRPALCIQKRHSSGSMDDRPSISARDYVESLHQNSRATLLYGKNNVLVQPRDDMEAVPGYLSLHQTADVMTLKWTPNQLMNGSVGDLDYEKSVYWDYAVTIRLEEIVYLHCHQQVDSGGTVVLVSQDGIQRPPFRFPKGGHLLQFLSCLENGLLPHGQLDPPLWSQRGKGKVFPKLRKRSPQGSSESTSSDKEDDEATDYVFRIIYPGTQSEFVPQDLMDVSMNNLPPLWQPSPRKSSCSSCSQSGSADGGSTNGCNHERAPLKLLCDNMKYQILSRAFYGWLAYCRHLSTVRTHLSALVNHMIVSPDLPCDAGQGLTASIWEKYIQDSTTYPEQELLRLIYYGGVQPEIRRAVWPFLLGHYQFGMTEMERKEVDEQIHACYAQTMSEWLGCEAIVRQRERESHAAALAKCSSGASLDSHLHRMLHRDSTISNESSQSCSSGRQNLRLQSDSSSSTQVFESVDEVEQTEAEGRSEEKHPKIPNGNPANGTCSPDSGHPSSHNFSSGLSEHSEPSLSTEDSVLDAQRSLPAVFRPGDSSVEDGQSSEATTSRDEAPREELAVQDSLESDLLANESLEEFMSIPGSLDVALPEKDGAVMDGWPGEADKPSRADSEDNLSEEPEMESLFPALASLAVTSSANNEASPVSSSGVTYSPELLDLYTVNLHRIEKDVQRCDRSYWYFTAANLEKLRNIMCSYIWQHIEIGYVQGMCDLLAPLLVILDDEALAFSCFTELMKRMNQNFPHGGAMDTHFANMRSLIQILDSELFELMHQNGDYTHFYFCYRWFLLDFKRELVYDDVFSVWETIWAAKHVSSAHYVLFIALALVEVYRDIILENNMDFTDIIKFFNEMAERHNAKQILQLARDLVHKVQILIENK

SEQ ID NO: 2 - DNA, 3282 bp, Mus musculus
atggcttcggtccctgcggaggccgagacccgccagaggctgctgcgcactgtgaagaaggaggtgaagcagatcatggaagaagccgttaccagaaagttcgtccacgaggacagcagccacatcatctccttctgtgcggccgtggaggcctgcgtgctgcacgggctgcggcggagggcggccggcttcctgcgaagcaacaagatcgcggcgctcttcatgaaggtgggcaaaggcttccctccggccgaggagctgagccgcaaggtccaggaactggagcaactcattgagagcgcgcgaaaccagatccagggcctgcaagagaacgtgcggaagctgccgaagctgcctaacctgtccccgctggccatcaagcacctgtggatccgcaccgccctgttcgagagggtcctggacaaaatcgtccactacctggtggaaaacagcagtaaatactacgagaaggaggcactcttgatggacccggtggacggacccatcctggcctctttgttggtggggccatgtgccctggagtataccaagatgaagactgcagatcacttctggactgacccctcagccgacgagctggtccagagacaccggatccacagctcacatctgagacaggactcgcccaccaagcggccagcgctctgtatccagaagaggcattccagtggcagcatggacgaccggccatctatctctgcccgggactatgtagaatctctgcaccagaactccagggccaccctgctctatggaaagaacaatgttctggttcagccaagggatgacatggaggccgtgccagggtacctgtctctgcaccagacagcggatgtcatgaccttgaagtggacacccaatcagctgatgaacgggtctgtgggggatctggactacgagaagagtgtctactgggactacgctgtgaccatccgcttagaggagatagtttacctgcactgccatcaacaagtggacagcggcgggactgtcgtgctggtgagccaggatggaatccagaggccacccttccgcttccccaagggcgggcacctgctacagttcctctcctgtctggagaatgggctgcttccgcacgggcagctggacccgccgctttggtcacagcgaggaaaggggaaggtatttcctaagttgcgcaagcgcagcccacaggggtcctccgagtccacgtcttcagacaaggaggacgacgaagccacggattacgtgttccgcatcatctaccctggcacgcagtctgaattcgtgccccaggacctaatggatgtctctatgaacaaccttccacccctatggcaacccagtcctcggaagtcctcctgctcttcctgttcacaaagcggctcagctgacggcggctcaaccaatggctgtaaccatgagagggccccactgaaactgctgtgtgataacatgaagtaccagatcctctccagagccttctatggatggctcgcctactgcagacacctgtccaccgtgaggacccacctgtcagccctggtcaatcacatgatcgtgtctccagacctgccctgtgacgctgggcaagggttgacagccagcatctgggaaaaatacatccaggacagcacgacctacccggagcaggagctgctgcgtctcatctactatggaggtgtccagcctgagatccgcagggcagtgtggcccttcctcctcggccactaccagtttgggatgacagagatggagaggaaagaggtagacgaacagatccatgcctgctacgcacaaaccatgtcggagtggctgggctgtgaagctatcgtgagacagagggagcgggagtcccacgcggctgccctggccaagtgctcttcaggagccagcctggacagccaccttcaccggatgctgcaccgggactccaccatcagcaatgagtcatcccagagctgcagctcaggccgccagaacctccgactgcagagcgactccagcagcagcacacaggtatttgagtctgtggatgaggtggagcagacagaggcagaaggcaggtcagaagaaaaacatcccaaaatccccaatgggaacccagccaacggcacttgctccccagactccggacacccttcctcccacaacttctcctctggtctctccgagcactctgagcccagtcttagcactgaagacagcgtcttggatgcccaacgcagccttccagctgtgtttcggcctggggacagcagcgtggaagatgggcagagcagcgaagccaccacatcccgggacgaggcccctcgggaggagctggccgtgcaagatagccttgagagtgacctccttgccaacgagagcttggaggagttcatgtctatccctggcagcctggacgtggccctacctgagaaggatggcgcggtgatggacggctggcccggtgaggcggacaagcccagtcgggctgacagtgaggacaacctctcagaagagcctgagatggagagcctgttccctgccctggcttctctggctgtgacctcctctgccaataacgaggcatcccctgtgtcctccagtggagtcacctattctccagaactactggacctgtacaccgtgaacctgcaccgcattgagaaagatgtgcagaggtgtgaccgcagctactggtacttcacagcagccaacctggagaagctgaggaacatcatgtgcagctatatctggcagcacatcgagatcggctacgtccagggcatgtgtgacctcctggccccgttgctggtcatcctggatgatgaggccctggccttcagttgcttcacggagctgatgaagaggatgaaccaaaacttcccccatggaggcgccatggatacgcacttcgccaacatgaggtctctgatccagatcctggactccgagctcttcgagctgatgcatcagaacggcgattacacccacttctacttttgctaccgctggttcctgctggatttcaaacgagagctggtctatgatgacgtcttctccgtgtgggaaactatctgggctgctaagcacgtgtcctccgcccactacgtgctgttcatcgcgctggcgctggtggaggtctacagagacatcatcctggagaacaatatggacttcactgacattatcaagttcttcaacgaaatggctgagcggcacaacgccaagcagattctgcagctcgcccgggacctcgtgcataaggtgcagattctaattgagaacaagtga

SEQ ID NO: 3 - protein, 16 aa, Mus musculus
CVMDGWPGEADKPSRA

SEQ ID NO: 4 - DNA, 34 bp, Artificial Sequence
  Nurr1 5′ Primer
agagtcgacggcagccatgccttgtgttcaggcg

SEQ ID NO: 5 - DNA, 35 bp, Artificial Sequence
  Nurr1 3′ Primer
ctaggcggccgcgggagaaggtcttagaaaggtaa

SEQ ID NO: 6 - DNA, 35 bp, Artificial Sequence
  NLBD 5′ Primer
tagagtcgacccaggatccctctcccccctcacct

SEQ ID NO: 7 - DNA, 35 bp, Artificial Sequence
  NLBD 583 3′ Primer
ctagcggccgcttatggtaccaagtcttccaattt

SEQ ID NO: 8 - DNA, 30 bp, Artificial Sequence
  RXR 5′ Primer
ctgggaattcacatggacaccaaacatttc

SEQ ID NO: 9 - DNA, 30 bp, Artificial Sequence
  RXR 3′ Primer
ctaagcggccgcctaagtcatttggtgcgg

SEQ ID NO: 10 - DNA, 38 bp, Artificial Sequence
  NuIP 5′ Primer
tagagtcgacggaaccgggcaccgaccagcttgagcca

SEQ ID NO: 11 - DNA, 31 bp, Artificial Sequence
  NuIP 3′ Primer
ctagtctagacttgttctcaattagaatctg

SEQ ID NO: 12 - DNA, 31 bp, Artificial Sequence
  VP16/NuIP 5′ Primer
tagagtcgacggcaccgaccagcttcagcca

SEQ ID NO: 13 - DNA, 32 bp, Artificial Sequence
  H1 5′ Primer
agagtcgaccgaagagcccacaggatccctct

SEQ ID NO: 14 - DNA, 34 bp, Artificial Sequence
  H1 3′ Primer
ctagtctagaatctccactcatctgatagtcagg

SEQ ID NO: 15 - DNA, 33 bp, Artificial Sequence
  H3 5′ Primer
tagagtcgacatgatacccaacatatccagcag

SEQ ID NO: 16 - DNA, 33 bp, Artificial Sequence
  H3 3′ Primer
ctagtctagagggagaaggtcttagaaaggtaa

SEQ ID NO: 17 - DNA, 22 bp, Artificial Sequence
  NuIP Forward Primer
ttgaagtggacacccaatcagc

SEQ ID NO: 18 - DNA, 24 bp, Artificial Sequence
  NuIP Reverse Primer
caggtctggagacacgatcatgtg

SEQ ID NO: 19 - DNA, 22 bp, Artificial Sequence
  NuIPa Forward Primer
ctctggcttcccacagtctccc

SEQ ID NO: 20 - DNA, 24 bp, Artificial Sequence
  NuIPa Reverse Primer
caggtctggagacacgatcatgtg

SEQ ID NO: 21 - DNA, 22 bp, Artificial Sequence
  NuIPa Reverse Primer
ctctggcttcccacagtctccc

SEQ ID NO: 22 - DNA, 22 bp, Artificial Sequence
  NuIPb Reverse Primer
gcttcttgcagacaacagcagg

SEQ ID NO: 23 - DNA, 22 bp, Artificial Sequence
  NuIPc Forward Primer
ttgaagtggacacccaatcagc

SEQ ID NO: 24 - DNA, 22 bp, Artificial Sequence
  NuIPc Reverse Primer
gcttcttgcagacaacagcagg

SEQ ID NO: 25 - DNA, 20 bp, Artificial Sequence
  Nurr1 Forward Primer
attccaatccggcaatgacc

SEQ ID NO: 26 - DNA, 21 bp, Artificial Sequence
  Nurr1 Reverse Primer
ttgcaacctgtgcaagaccac

SEQ ID NO: 27 - DNA, 19 bp, Artificial Sequence
  NuIP siRNA 1
gtaccagatcctctccaga

SEQ ID NO: 28 - DNA, 19 bp, Artificial Sequence
  NuIP siRNA 2
cccgggacctcgtgcataa

SEQ ID NO: 29 - protein, 146 aa, Mus musculus
SFCAAVEACVLHGLRRRAAGFLRSNKIAALFMKVGKGFPPAEELSRKVQELEQLIESARNQIQGLQENVRKLPKLPNLSPLAIKHLWIRTALFERVLDKIVHYLVENSSKYYEKEALLMDPVDGPILASLLVGPCALEYTKMKTAD

SEQ ID NO: 30 - protein, 173 aa, Mus musculus
LHRIEKDVQRCDRSYWYFTAANLEKLRNIMCSYIWQHIEIGYVQGMCDLLAPLLVILDDEALAFSCFTELMKRMNQNFPHGGAMDTHFANMRSLIQILDSELFELMHQNGDYTHFYFCYRWFLLDFKRELVYDDVFSVWETIWAAKHVSSAHYVLFIALALVEVYRDIILENN

SEQ ID NO: 31 - protein, 598 aa, Mus musculus
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPSFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 32 - protein, 245 aa, Mus musculus
DPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIDKLFLDTLPF

SEQ ID NO: 33 - protein, 230 aa, Mus musculus
DPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVP

SEQ ID NO: 34 - protein, 598 aa, Mus musculus
MPCVQAQYGSSPQGASPASQSYSYHSSGEYSSDFLTPEFVKFSMDLTNTEITATTSLPSFSTFMDNYSTGYDVKPPCLYQMPLSGQQSSIKVEDIQMHNYQQHSHLPPQSEEMMPHSGSVYYKPSSPPTPSTPSFQVQHSPMWDDPGSLHNFHQNYVATTHMIEQRKTPVSRLSLFSFKQSPPGTPVSSCQMRFDGPLHVPMNPEPAGSHHVVDGQTFAVPNPIRKPASMGFPGLQIGHASQLLDTQVPSPPSRGSPSNEGLCAVCGDNAACQHYGVRTCEGCKGFFKRTVQKNAKYVCLANKNCPVDKRRRNRCQYCRFQKCLAVGMVKEVVRTDSLKGRRGRLPSKPKSPQDPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIAKLFLDTLPF

SEQ ID NO: 35 - protein, 245 aa, Mus musculus
DPSPPSPPVSLISALVRAHVDSNPAMTSLDYSRFQANPDYQMSGDDTQHIQQFYDLLTGSMEIIRGWAEKIPGFADLPKADQDLLFESAFLELFVLRLAYRSNPVEGKLIFCNGVVLHRLQCVRGFGEWIDSIVEFSSNLQNMNIDISAFSCIAALAMVTERHGLKEPKRVEELQNKIVNCLKDHVTFNNGGLNRPNYLSKLLGKLPELRTLCTQGLQRIFYLKLEDLVPPPAIIAKLFLDTLPF